(19) United States
(12) Patent Application Publication
(10) Pub. No.: US 2018/0325406 A1
(43) Pub. Date: Nov. 15, 2018

(54) RAPID EKG LEAD PLACEMENT DEVICE

(71) Applicants: Joseph Wayne Kraft, College Station, TX (US); Scott Lawson Beckwith, Dallas, TX (US)

(72) Inventors: Joseph Wayne Kraft, College Station, TX (US); Scott Lawson Beckwith, Dallas, TX (US)

(21) Appl. No.: 15/593,808

(22) Filed: May 12, 2017

(51) Int. Cl. A61B 5/0408

(52) U.S. Cl. CPC A61B 5/04085; A61B 5/04087

(57) ABSTRACT

This device is designed to significantly improve the diagnostic value of Electrocardiography by reducing the time for placement of Electrocardiogram (EKG) leads on a patient and reducing technician variability in performing EKGs. By utilizing an elastomeric harness to which EKG leads can be pre-attached, the harness can be applied to the patient very rapidly and provide faster and more reliable information for the patient's care.

### PRIORITY

This applications claims priority from the disclosure of U.S. Provisional Patent Application No. 62/387,825, which is incorporated by reference herein.

### FIELD OF INVENTION

This device refers to the field of medical devices and more particularly to electrocardiography.

### BACKGROUND OF THE INVENTION

An electrocardiogram (EKG) is a medical test for evaluating electrical conduction through cardiac muscle. It is performed very commonly in both the inpatient and outpatient settings to evaluate a patient for many different cardiac maladies including, but not limited to myocardial infarction (heart attack) and cardiac arrhythmias. In order to perform an EKG with currently available technology, at least twelve wires are connected to specific locations on the patient using small electrically conductive adhesive patches to which the wires are attached. Currently available techniques to attach EKG leads to a patient are slow, confusing, cumbersome and problematic in many situations mainly due to the fact that each lead is attached individually. The information from the EKG is available immediately and used to diagnose and treat patients with life-threatening conditions and therefore if the procedure itself is time consuming or yields erroneous information then patients can be at serious risk of significant morbidity and mortality. This proposed device is designed to be a harness that makes attaching the EKG leads to the patient much more expedient, consistent and safe.

### SUMMARY OF THE INVENTION

This proposed device is designed to significantly decrease the time it takes to perform the potentially life saving medical test called an Electrocardiogram (“EKG”). Current practice for a patient with a cardiac complaint is to immediately perform an EKG to rule out life many life-threatening conditions, which could require immediate intervention. Current technology requires that the caregiver must individually remove the adhesive backing from each of the twelve adhesive electroconductive contact pads and strategically place each contact pad onto the patient's chest wall in locations that will best evaluate cardiac electrical conductivity through a multitude of axes. Then each of the twelve electrode leads is untangled and each specific lead is identified and attached to its specific anatomically matched adhesive contact pad.

With this proposed device the time required to perform the EKG is significantly reduced because instead of having to individually apply twelve separate wires (leads), each with an electroconductive adhesive contact pad, this device has all the leads pre-wired into an specifically shaped elastomeric harness that can be quickly wrapped around a patient's chest thus holding the contacts tight against the skin. With the harness being elastomeric the device can be stretched to fit multiple different body sizes and shapes. This also will help to hold the contacts against the skin without having to depend on the adhesive property of the traditional electroconductive contact, which can be problematic in patients who may be diaphoretic or if they have a lot of chest hair, which could prevent adequate adhesion of the contact pad to the skin. In an envisioned scenario, the harness with the pre-attached leads in place can be laid out on the stretcher in the emergency department or ambulance and be ready for rapid deployment should a patient with a cardiac problem present. This would certainly save significant time and would allow for more standardization of the EKG by removing technician variability in electrode lead placement.

Another significant improvement in this device is that it can be easily utilized to more thoroughly evaluate a patient that may be having cardiac ischemia or infarction (heart attack) of the right side or posterior wall of the heart. Many times a traditional “left-sided” EKG can miss an acute myocardial infarction if the area of infarction is located in one of the “blind spots” on right side or posterior aspect of the heart. When this type of infarction is suspected and the traditional EKG is non-diagnostic, then the wise clinician will likely order a “right-sided” EKG where the leads are removed from the left side of the chest and new electroconductive pads with attached electrodes are place around the right side of the chest. This proposed device can have all of the left as well as right-sided contacts already in place and therefore the clinician can have access to a complete electrocardiographic assessment of the heart by simply “flipping a switch” to convert the EKG machine from a standard left-sided EKG to a right sided EKG. This would greatly reduce the chance of missing the diagnosis of a potentially life threatening posterior wall or right ventricular myocardial infarction. This simple and easy expansion of the diagnostic capabilities of the EKG could very easily reduce the potential for adverse outcomes in morbidity and mortality.

### BRIEF DESCRIPTION OF THE DRAWINGS

Without restricting the full scope of this invention, the preferred form of this invention is illustrated in the following drawings:

FIG. 1 displays a patient with traditional EKG leads in place; and

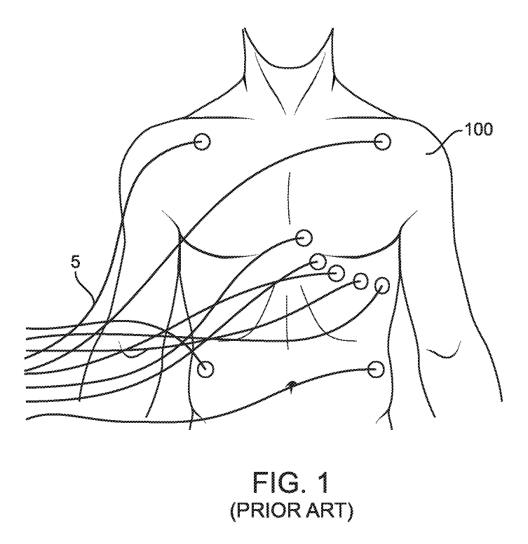

FIG. 2 shows a patient with one embodiment of the proposed EKG lead harness in place.

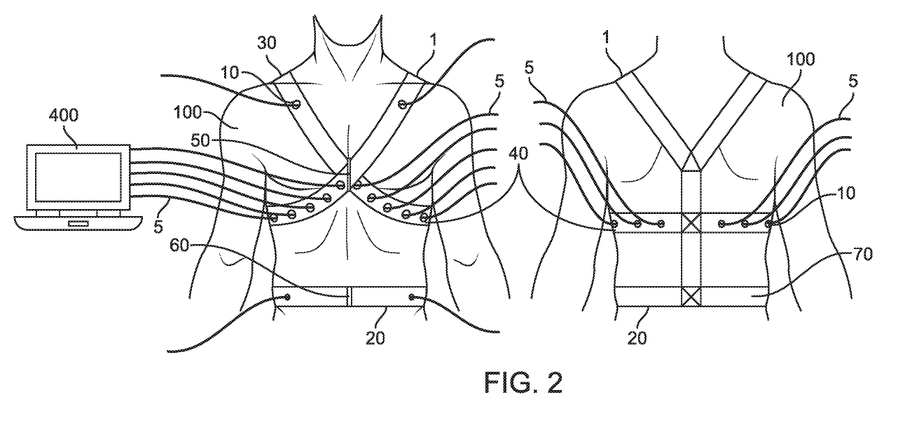

### DETAILED DESCRIPTION OF PREFERRED EMBODIMENTS

Electrocardiograms are frequently performed in an emergent situation where time is of the essence and a patient's life is at stake. They are very frequently utilized in Emergency Rooms, clinics and hospitals to identify many different cardiac pathologies. This is the most commonly employed cardiac evaluation performed today. When a patient 100 presents with cardiac symptoms, a technician or nurse is usually tasked with the arduous process of performing an Electrocardiogram (“EKG”) utilizing twelve wires 5 (leads). The first step is to untangle the wires 5 that correspond to each of the twelve EKG leads. Then each of the wires 5 is attached to an adhesive pad 15 that is used to connect the EKG lead to the patient's 100 chest wall. Then the technician places each adhesive pad 15/lead 5 combination onto the patient 100 in predetermined locations on the chest wall to best evaluate the electrical conduction through the patient's heart. When it is determined by the EKG analyzer that the leads are all making adequate contact then the EKG analyzer can run and print the EKG to make it immediately available for clinical interpretation and patient management.

Due to the fact that there is a wide range of patient body morphologies, ranging from young to old and from thin to morbidly obese, this process is very time consuming and wrought with a wide range of user variability and frustration, thus leading to much less consistent and reliable data for the clinician to use to make the proper diagnosis. Anything that can delay or alter the proper diagnosis has great potential to have adverse patient outcomes including, quite often, death from an improperly diagnosed cardiac event.

This proposed device reduces both the procedural time and the user variability thus producing a much more consistent and useful diagnostic tool. By using an elastomeric harness 1 the device can be placed on different sizes and shapes of patients 100 just by stretching to fit around the patient's chest wall. The harness 1 can be of any design or configuration. It can be designed to be a single pull over piece or a single piece with connectors like buckles or hook and loop fasteners or multiple pieces like a chest strap and belt. It can be designed to be used with all the EKG leads and their proper position or a subset.

In one embodiment, the harness 1 would have a belt section 20 around a patient's waist, shoulder straps 30 across a patient's shoulders, and a chest strap 40 around a patient's chest. The shoulder straps 30 and chest straps 40 would connect at a patient's sternum area with a chest connecting means 50 such as, but not limited to, a hook and loop connector. The belt 20 would also have a fastening means 60 to secure it to the patient 100. The harness 1 would have a base 70 in the back to which the straps and belt of the harness are connected.

The harness 1 could be available in a few different sizes to account for the extremes in patient sizes and these could be differentiated by color.

The harness 1 has strategically placed electroconductive contacts 10 which penetrate through the elastomeric material thus providing a patient contact surface on the inner aspect of the harness and a lead attachment site on an outward surface. These contacts 10 will be distributed in such a way so as to properly evaluate the electrical conduction through the cardiac muscle. Each contact will have an attachment site for pre-attachment of the EKG leads/wires 5 to go directly to the EKG analyzer. The harness 1 with the pre-attached EKG leads can be prepared ahead of time to be ready for immediate application to a patient when a patient presents with cardiac symptoms. The elastomeric nature of this device also has the added advantage that there would be fewer problems with lead non-adherence due to skin moisture, body hair, or topical lotions.

This proposed device will also have the additional advantage of having an extra set of right sided EKG leads already in place in order to be able to immediately perform a right-sided EKG to rule out posterior or right ventricular infarction which, unfortunately often goes undiagnosed if not specifically evaluated. In one embodiment there can be a simple incorporated switch to alternate between a standard left-sided EKG and a right-sided EKG without having to individually place the additional leads. This can be performed very quickly as serial EKGs using the exact same EKG analyzer and just flipping a switch between exams to switch from the traditional leads to the right-sided leads. This will have the added advantage that medical facilities can utilize the currently available standard EKG analyzing equipment to perform both exams rapidly and with much less technician-to-technician variability.

1-7. (canceled)

8. A device comprising;
a colored elastic harness where the color is based on its size with attached electroconductive contacts where the contacts will have an attachment site for the pre-attachment of an electrocardiogram (EKG) lead where the leads are placed for an electrocardiogram reading where the harness is comprised of a belt section, shoulder straps, and a chest strap, where the shoulder straps and chest straps connect with the electroconductive contacts for both left and right-sided EKG data collection performed in series utilizing readily available EKG analyzers, having a back with a base where the shoulder straps and belt section of the harness are connected.

9. A device according to claim 8 comprising: where the belt section has a fastening means.

10. A device according to claim 8 comprising an inner aspect of the harness and a lead attachment site on an outward surface of the harness having the electroconductive contacts penetrate through the harness to connect to a contact surface.
